(19) United States
(12) Patent Application Publication
(10) Pub. No.: US 2005/0015080 A1
(43) Pub. Date: Jan. 20, 2005

(54) Device for cutting or heating medical implants

(76) Inventors: Paul Ciccone, Lincoln University, PA (US); Emmanuel Vander Vennen, Gibbstown, NJ (US); Michael C. Chen, Philadelphia, PA (US)

Correspondence Address: JONES DAY, 222 EAST 41ST ST, NEW YORK, NY, 10017, US

(21) Appl. No.: 10/893,864

(22) Filed: Jul. 15, 2004

Related U.S. Application Data
(60) Provisional application No. 60/487,885, filed on Jul. 16, 2003.

(51) Int. Cl. A61B 18/04; H05B 1/00; H05B 3/42; H01L 21/26; H01L 21/42

(52) U.S. Cl. 606/30; 219/233; 219/238

(57) ABSTRACT

The present invention relates to a hand-held device for cutting or heating medical implants, in particular for cutting and bending resorbable implants. The device comprises a heating element electrically connectable to a power source (e.g., a battery), a switch for selectively completing the circuit between the heating element and the power source, and a housing for enclosing the battery and associated electrical connections. The invention is directed to a kit comprising a handle assembly and multiple interchangeable cutting or heating tips for use therewith. One tip member is configured for heating an implant to a sufficient temperature to cut the implant while a second tip member is configured for heating the implant to a sufficient temperature to bend the implant. A tip member housing an additional power source may be provided to bend larger size implants.

### CROSS-REFERENCE TO RELATED APPLICATIONS

This application claims the benefit of U.S. Provisional Application No. 60/487,885, filed Jul. 16, 2003.

### FIELD OF THE INVENTION

The invention relates to hand-held device for cutting or heating medical implants, in particular the invention relates to an in-situ bender cutter device for resorbable implants.

### BACKGROUND OF THE INVENTION

In the field of medical instruments, various hand-held instruments for heating exist. For instance, hand-held instruments exist for cauterizing tissue of a patient in order to prevent or stop bleeding from blood vessels.

A typical instrument used for heating in the field of medical instruments comprises a heating element electrically connectable to a power source (e.g., a battery), a switch for selectively completing the circuit between the heating element and the power source, and a housing for enclosing the battery and associated electrical connections. The electrical connections typically comprise a strip of conductive material connecting one end of the heating element to a positive terminal of the power source and another strip of conductive material connecting the other end of the heating element to a negative terminal of the power source with the switch providing a break therebetween. The electrical circuit of the heating instrument is normally open and it does not become a completed circuit unless pressure is applied to a movable member (i.e. a button) on the exterior of the housing to engage the switch. Typically, the heating element is fixedly attached to the housing and changing the types of heating elements attached to the housing is difficult without destroying the utility of the instrument.

A need exists for an improved heating instrument with interchangeable heating elements or tips.

### SUMMARY OF THE INVENTION

The present invention generally relates to a device for use with a resorbable fixation system including a resorbable implant. In one embodiment, the invention is directed to a kit comprising a handle assembly and three interchangeable tips for use therewith. The handle assembly comprises a longitudinal member extending from a proximal end to a distal end and houses a power source. The kit also includes a first tip member removably attachable to the handle assembly and comprises a first heating element electrically coupleable to the power source. The first heating element is configured and dimensioned for heating an implant to a sufficient temperature to cut the implant when the first heating element is electrically coupled to the power source while being applied to the implant. A second tip member is included in the kit and is removably attachable to the handle assembly and comprises a second heating element electrically coupleable to the power source, and the second heating element is configured and dimensioned for heating the implant to a sufficient temperature to bend the implant when the second heating element is electrically coupled to the power source while being applied to the implant. Also included in the kit is a third tip member that is removably attachable to the handle assembly. The third tip member houses an additional power source and comprises a third heating element electrically coupleable in series to both the internal power source and the power source located in the handle assembly, and the third heating element is configured and dimensioned for heating the implant to a sufficient temperature to bend the implant when the third heating element is electrically coupled to the power sources while being applied to the implant. The first heating element comprises a first surface area, the second heating element comprises a second surface area, and the third heating element comprises a third surface area, and the first, second, and third surface areas are different and are configured and dimensioned to heat to different temperatures when electrically coupled to the power source.

### BRIEF DESCRIPTION OF THE DRAWINGS

Preferred features of the present invention are disclosed in the accompanying drawings, wherein similar reference characters denote similar elements throughout the several views, and wherein:

FIG. 1 is a perspective view of a preferred embodiment of a hand-held device according the invention;

FIG. 2 is a top view of the hand assembly of FIG. 1;

FIG. 3 is a cross-sectional view along line III-III of FIG. 2;

FIG. 4 is a top view of a preferred embodiment of a tip member for use with the handle assembly of FIG. 1;

FIG. 5 is a side view of the tip member of FIG. 4;

FIG. 6 is a top view of a preferred embodiment of another tip member for use with the handle assembly of FIG. 1;

FIG. 7 is a side view of the tip member of FIG. 6;

FIG. 8 is a top view of a preferred embodiment of yet another tip member for use with the handle assembly of FIG. 1;

FIG. 9 is a side view of the tip member of FIG. 8; and

FIG. 10 is a cross-sectional view along line IV-IV of FIG. 8.

FIG. 11 is a cross-sectional view along line A-A of the heating element of FIG. 10.

### DETAILED DESCRIPTION OF THE PREFERRED EMBODIMENTS

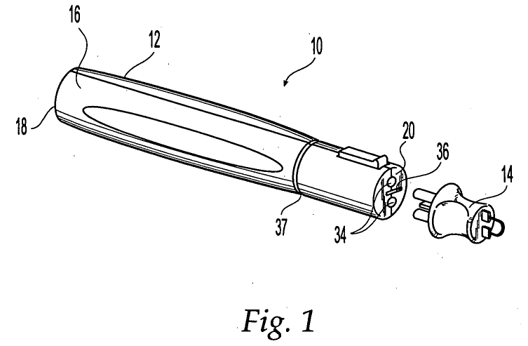

Referring to FIG. 1, a preferred embodiment of a hand-held device 10 according to the present invention is shown. Hand-held device 10, comprises a handle assembly 12 that mates with at least one removable tip member 14. Handle assembly 12 comprises a generally cylindrical outer housing 16 extending from a proximal end 18 to a distal end 20, and tip member 14 is removably attachable to distal end 20.

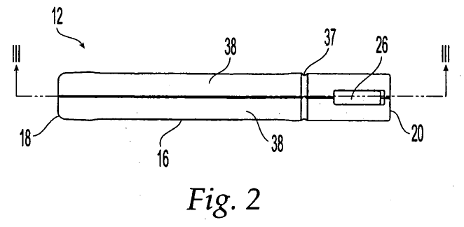

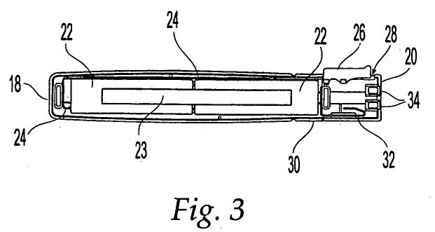

Referring to FIGS. 2-3, housing 16 includes a power source. For example, in a preferred embodiment at least one battery 22 is disposed in housing 16 toward distal end 20, and preferably two batteries 22 are provided. Throughout the following discussion, batteries 22 may be referred to in the singular or plural, however, it will be recognized that all of the embodiments disclosed herein may be implemented using either one or two batteries. As shown in FIG. 3, batteries 22 may be secured with tape 23 to diminish rattling. A conductor 24 contacts battery 22 adjacent proximal end 18 and extends within housing 16 from proximal end 18 toward distal end 20 to selectively couple battery 22 to tip member 14. A selectively depressible power button 26 is coupled to the distal end of conductor 24 adjacent distal end 20, and extends through a slot 28 in outer housing 16. In one preferred embodiment, conductor 24 comprises a thin metal strip and power button 26 can be ultrasonically welded to an outside face of conductor 24. Power button 26 is selectively depressible between a rest position and a second position to flex the distal end of conductor 24 to engage tip member 14 and electrically connect tip member 14 to battery 22. A second conductor 30 contacts the distal end of battery 22 and includes a free end 32 that extends radially inward to couple the distal end of battery 22 to tip member 14. As best seen in FIG. 1, distal end 20 of handle assembly 12 includes a pair of circular socket holes 34 and a generally rectangular slot 36 for receiving tip member 14. In a preferred embodiment, power button 26 and housing 16 are made of plastic and housing 16 preferably comprises shell members 38 ultrasonically welded together.

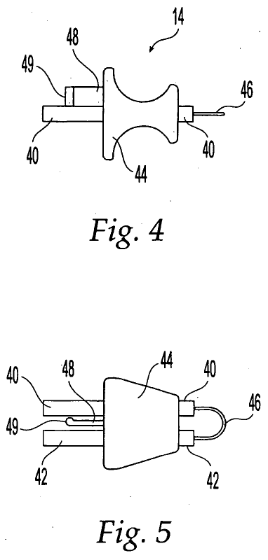

Referring to FIGS. 4-5, one preferred tip member 14 comprises a cutting tip member and includes first and second conductive prongs 40, 42 extending from a proximal end of a tip body 44. Prongs 40, 42 are electrically coupled to opposing ends of a heating element 46 that extends from the distal end of tip body 44. A rectangular tab 48 extends proximally from tip body 44 and is configured and dimensioned to be received in slot 36 (shown in FIG. 1) of handle assembly 12 for aligning tip body 44 with the handle assembly when tip member 14 is attached to the handle assembly. In a preferred embodiment, tab 48 includes a protrusion 49 along its proximal end and when tab 48 is inserted into rectangular slot 36, the proximal end of tab 48 extends through handle housing 16 and protrusion 49 engages the interior of housing 16 to retain tip member 14 on the handle assembly 12.

As best seen in FIG. 5, tip body 44 is tapered from a proximal end to a narrower distal end and comprises concave lateral cutout portions on the sides to accommodate, for example, a user's fingers to facilitate attachment of the tip to handle assembly 12. Prongs 40, 42 are configured and dimensioned to be received in socket holes 34 when tip member 14 is attached to handle assembly 12. Prongs 40, 42 extend through socket holes 34 and into handle assembly 12 and second prong 42 contacts free end 32 of second conductor 30. Power from battery 22 may be transferred to heating element 46 by depressing power button 26 to flex conductor 24 to engage first prong 40 and complete an electric circuit. Heating element 46 generally comprises a thin wire loop that heats up when an electric current passes through it. The thin wire loop has a relatively small surface area which facilitates efficient heating of material. When heated, the wire loop is configured and dimensioned to operate at a temperature sufficient to cut through a medical implant, and in particular a resorbable implant. In use, the implant may be cut by contacting the heating element 46 to the area of the implant to be cut with power button 26 depressed.

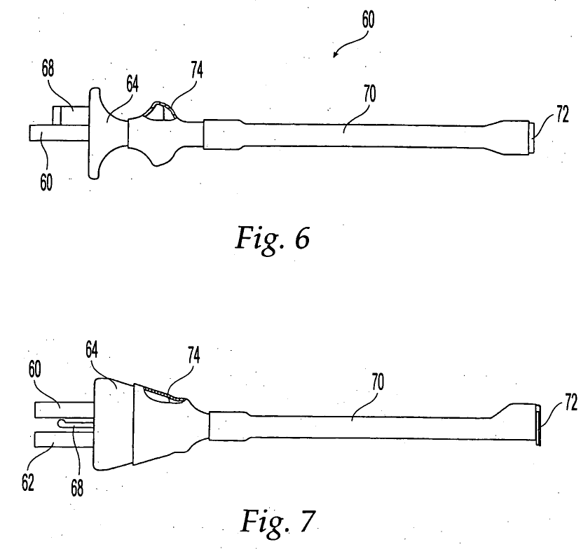

Referring to FIGS. 6-7, another preferred tip member 60 comprises a narrow plate bending tip member. The proximal end of tip member 60 is substantially similar to the proximal end of tip member 14 and comprises first and second conductive prongs 60, 62 and a rectangular tab 68 extending from a proximal end of a tip body 64 to attach to the distal end of handle assembly 12 as described above with respect to tip member 14. A generally cylindrical elongate flexible member 70 extends from the distal end of tip body 64 and includes a heating element 72 at the distal end. Flexible member 70 comprises an insulating material covering a conductive element that electrically couples heating element 72 to prongs 60, 62. In a preferred embodiment, a protective covering 74 extends over a portion of tip body 64 and over a portion of elongate member 70 to cover and insulate the electrical connection between prongs 60, 62 and the conductive element extending through elongate member 70. Heating element 72 generally comprises a flat narrow metal plate or bending element. Power from battery 22 may be transferred to heating element 72 when tip member 60 is attached to handle assembly 12, as described above. The flat narrow plate generally has larger surface area than heating element 46, described above, and heating element 72 is generally heated to a lower temperature relative to heating element 46. In operation, heating element 72 is configured and dimensioned to operate at a temperature sufficient to bend or form the implant. For example, the implant may be formed by contacting the heating element 72 to the area of the implant to be bent with power button 26 depressed. Once sufficiently heated, the heated area of the implant remains malleable for a period of time and the implant may be contoured to a desired geometry.

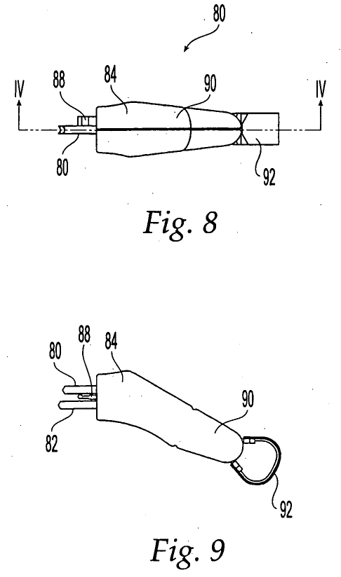

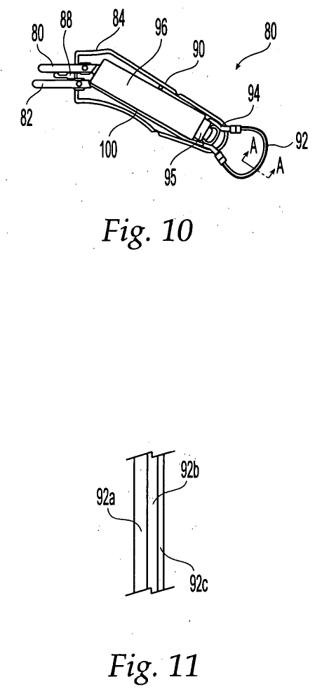

Referring to FIGS. 8-11, another preferred tip member 80 comprises a wide plate bending tip member. The proximal end of tip member 80 is substantially similar to the proximal end of tip members 14, 60 described above and comprises first and second prongs 80, 82 and a rectangular tab 88 extending from a proximal end of a tip body 84 to attach to the distal end of handle assembly 12, as described above. An elongate member 90 extends from the distal end of tip body 84 and includes a heating element 92 at the distal end. Elongate member 90 extends at an angle with respect to handle assembly 12 to facilitate application of heating element 92 to an implant. Heating element 92 generally comprises a large flexible loop with a generally flat wide section in the central portion.

As illustrated in FIG. 11, the flexible loop may have a composite structure comprising a bottom layer of foam 92a, a flexible heater layer comprising a plastic flexible circuit 92b (i.e. a plurality of copper tracing sandwiched between layers of plastic), and a top layer of metal foil 92c. The bottom foam layer 92a may provide structural support for the plastic flexible circuit 92b, while the top metal foil layer 92c may spread the heat generated by the flexible circuit evenly over the surface of the heating element 92. A battery 96 is disposed in housing 90 towards the distal end to provide additional current to heating element 92. A conductor 94 contacts a coil spring 95 which in turn contacts the distal end of battery 96 adjacent distal end and extends within housing 90, towards heating element 92, selectively coupling battery 96 to heating element 92. A second conductor 100 contacts the distal end of prong 82 and extends within housing 90, towards heating element 92 selectively coupling prong 82 to heating element 92. The proximal end of battery 96 extends to distal end of prong 80, selectively coupling battery 96 with prong 80. Power from battery 22 and battery 96 may be transferred to heating element 92 when tip member 80 is attached to handle assembly 12, and power button 26 is depressed, as described above. The large metal loop of heating element 92 generally has a larger surface area than heating elements 46 and 72, described above. This larger surface area allows the user to bend or form larger implants than with heating element 72. In operation, heating element 92 is configured and dimensioned to operate at a temperature sufficient to bend or form an implant as described above with respect to heating element 72, and is particularly well suited for bending or forming a resorbable mesh.

In operation, hand-held device 10 may be used in heating, forming or cutting medical implants, such as plates, meshes, sheets, screws, and tacks. For instance, device 10 may be used before, during, and after surgery, in-situ or otherwise. In particular, device 10 may be used with resorbable fixation systems such as those used in maxillofacial applications. To cut an implant, the cutting tip member 14 may be attached to distal end 20 of handle assembly 12. In this regard, rectangular tab 48 should be aligned with slot 36 for aligning tip body 44 with handle assembly 12 when tip member 14 is attached to handle assembly 12. In use, power button 26 may be depressed for approximately 3-5 seconds to heat up heating element 46. While keeping power button 26 depressed, heating element 46 may be applied to the area of the implant to be cut. After the implant has been cut, tip member 14 can be removed from the handle and other tip members can be attached to handle assembly 12.

The heat an implant, such as a resorbable plate, for forming or bending the implant, the narrow plate bending tip member 60 or the wide plate bending tip member 80 may be attached to distal end 20 of handle assembly 12. In use, power button 26 may be depressed for approximately 5-10 seconds to heat up heating element 72, or approximately 10-15 seconds to heat up heating element 92. While keeping power button 26 depressed, heating element 72, 92 may be applied to the area of the implant to be heated for forming or bending. In a preferred embodiment, the heating element 72, 92 is applied to the implant for approximately 10-15 seconds. After the implant has been sufficiently heated, the heated area will generally remain malleable for a period of time, during which the implant can be contoured to a desired geometry. In a preferred embodiment, the implant remains malleable for approximately 7-10 seconds. After the implant is contoured to the desired geometry it may be allowed to cool to original rigidity. To contour additional areas of the implant the process can be repeated.

Referring again to FIG. 1, device 10 may be a single use device that is disposable after use. In a preferred embodiment, a stress channel 37 extends around the circumference of outer housing 16 proximal to power button 26. If desired, prior to disposal of handle assembly 12, the batteries 22 within handle assembly 12 can be removed for reuse or separate disposal by cracking housing 16 open at stress channel 37.

As will be appreciated, the tip members described above can be interchangeably attached to handle assembly 12. Such interchangeability advantageously provides the user an option to use different tip members as appropriate, depending on the type of implant to be cut, formed, contoured, or otherwise heated, particularly in the case of resorbable implants. For example, cutting tip member 14 is generally well suited for cutting implants such as resorbable plates, and screws. Narrow plate bending tip 60 is generally well suited for forming or bending resorbable plates and wide plate bending tip member 80 may be more appropriate for use to form or bend a resorbable mesh material. In this regard, a kit for use with a resorbable fixation system may be provided that comprises a handle assembly 12, cutting tip member 14, narrow plate bending tip member 60, and wide plate bending tip member 80. In a preferred kit, a handle assembly 12 will be provided, as will one cutting tip member 14, and either a narrow or wide plate bending tip member 60, 80.

While various descriptions of the present invention are described above, it should be understood that the various features can be used singly or in any combination thereof. Therefore, this invention is not to be limited to only the specifically preferred embodiments depicted herein. Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

1. A device for cutting or bending medical implants comprising:
a handle comprising a power source;
a first tip member removably attachable to the handle and comprising a first heating element electrically coupleable to the power source, the first heating element configured and dimensioned for heating an implant to a sufficient temperature to cut the implant when the first heating element is electrically coupled to the power source while engaging the implant;
a second tip member removably attachable to the handle and comprising a second heating element electrically coupleable to the power source, the second heating element configured and dimensioned for heating an implant to a sufficient temperature to bend the implant when the second heating element is electrically coupled to the power source at least while placed near the implant;
wherein the first and second heating elements comprise first and second surface areas, respectively, which are configured and dimensioned to heat to different temperatures when electrically coupled to the power source.

2. The device of claim 1, wherein the handle includes socket holes for receiving prongs of a tip member.

3. The device of claim 1, wherein the handle assembly includes a power button that is selectively depressible between a rest position and a second position to electrically couple the power source to the first or second heating element.

4. The device of claim 1, wherein the first heating element comprises a wire loop able to be heated when an electric circuit passes through it.

5. The device of claim 1, wherein the second heating element comprises a generally flat plate.

6. The device of claim 1, wherein the second surface area is greater than the first surface area.

7. The device of claim 1, wherein the first heating element may be heated to a higher temperature than the second heating element when coupled to the power source.

8. The device of claim 1, further comprising a third tip member removably attachable to the handle and comprising a supplemental internal power source coupleable to the handle power source, the third heating element configured and dimensioned for heating the implant to a sufficient temperature to bend the implant when the third heating element is electrically coupled to the supplementary and handle power sources while placed near the implant.

9. The device of claim 8, wherein the third heating element comprises a third surface area that is different from the first and second surface areas.

10. The device of claim 8, wherein the third heating element is configured to heat to a different temperature than the first and second heating elements when electrically coupled to the supplementary and handle power sources.

11. The device of claim 8, wherein the third heating element comprises a flat metal loop.

12. A kit for use with a resorbable fixation system including at least one resorbable implant, the kit comprising:
a handle assembly comprising a longitudinal member extending from a proximal end to a distal end and housing a primary power source;
a first tip member removably attachable to the handle assembly and comprising a first heating element electrically coupleable to the power source, and the first heating element configured and dimensioned for heating an implant to a sufficient temperature to cut the implant when the first heating element is electrically coupled to the primary power source while engaging the implant;
a second tip member removably attachable to the handle assembly and comprising a second heating element electrically coupleable to the power source, and the second heating element configured and dimensioned for heating an implant to a sufficient temperature to bend the implant when the second heating element is electrically coupled to the primary power source at least while placed near the implant; and
a third tip member removably attachable to the handle assembly and comprising a third heating element and a supplementary internal power source, both electrically coupleable to the primary power source, the third heating element configured and dimensioned for heating an implant to a sufficient temperature to bend the implant when the third heating element is electrically coupled to the supplementary and primary power sources at least while placed near the implant;
wherein the first heating element comprises a first surface area, the second heating element comprises a second surface area, and the third heating element comprises a third surface area, and wherein the first, second, and third surface areas are different and are configured and dimensioned to heat to different temperatures when electrically coupled to the power source.

13. The kit of claim 12, wherein handle assembly includes socket holes for receiving prongs of a tip member.

14. The kit of claim 12, wherein the handle assembly includes a power button that is selectively depressible between a rest position and a second position to electrically couple the power source to a heating element.

15. The kit of claim 12, wherein the first heating element comprises a wire loop able to be heated when an electric circuit passes through it.

16. The kit of claim 12, wherein the second heating element comprises a generally flat plate.

17. The kit of claim 12, wherein the second surface area is greater than the first surface area.

18. The kit of claim 12, wherein the first heating element may be heated to a higher temperature than the second heating element when coupled to the power source.

19. The kit of claim 12, wherein the third heating element comprises a flat metal loop.

20. The kit of claim 12, wherein the third surface area is greater than the second surface area.

21. The kit of claim 12, wherein the second heating element may be heated to a higher temperature than the third heating element when coupled to the power source.
